(19) United States
(12) Patent Application Publication
(10) Pub. No.: US 2010/0049122 A1
(43) Pub. Date: Feb. 25, 2010

(54) Manual Breast Pump

(75) Inventors: Reinhold Jaeger-Waldau, Scheessel (DE); Guenter Marr, Rosengarten (DE)

Correspondence Address: VIDAS, ARRETT & STEINKRAUS, P.A., SUITE 400, 6640 SHADY OAK ROAD, EDEN PRAIRIE, MN, 55344, US

(73) Assignee: MAPA GMBH, Zeven (DE)

(21) Appl. No.: 12/516,148

(22) PCT Filed: Sep. 29, 2007
(86) PCT No.: PCT/EP2007/008501

§ 371 Date: May 22, 2009

(30) Foreign Application Priority Data
Nov. 24, 2006 (DE) 10 2006 056 321.2

(51) Int. Cl. A61M 1/06

(52) U.S. Cl. 604/74

(57) ABSTRACT

A breast pump for pumping down breast milk, with a cylindrical hollow piston with at least one deepening circulating on the outside on the lower end with respect to the cylindrical superficies surface of the hollow piston, a suction cup, which is connected to the tipper end of the hollow piston via a passage for fluid, a soft elastic compensation element (8) between the hollow piston and the bearing surface of the suction cup, at least one circulating sealing element made of a soft elastic material, inserted into the deepening, and a cylinder with a closed lower end, in which the hollow piston is axially movable, wherein the superficies surface of the hollow piston is guided on an inner wall of the cylinder, the sealing element is sealingly arranged between the inner wall of the cylinder and the hollow piston, and at least the suction cup is arranged outside of the cylinder.

### CROSS-REFERENCE TO RELATED APPLICATIONS

Not applicable

### STATEMENT REGARDING FEDERALLY SPONSORED RESEARCH

Not applicable

### BACKGROUND OF THE INVENTION

The present invention is related to a manually operable breast pump for pumping down breast milk.

Breast pumps have at least one suction cup, which is set on the mother's breast. A negative pressure is applied to the Suction Cup in order to withdraw milk from the mother's breast. The suction cup is connected to a container which receives the breast milk which was sucked down.

From EP 0 11 6 186 A1 a manually operable breast pump is known, which has a longitudinal outer cylinder with a closed lower end and a longitudinal pipe-shaped portion or hollow piston, respectively, which is connected integrally with a suction cup on its upper end, and detachably with a sealing ring on its lower end. The pipe-shaped portion is movably arranged inside the outer cylinder, in order to obtain a suction effect when the suction cup is set on a breast. The sealing ring has a lip projecting towards the upside which in fact seals well when aspirating, but via which milk can flow over when the pipe-shaped portion and the outer cylinder are thrust together. The longitudinal pipe-shaped portion tapers, so that its outer diameter is smaller in the vicinity of the suction cup than the outer diameter in the vicinity of its lower end, in order to permit a tilting of the longitudinal axis of the outer cylinder relative to the longitudinal axis of the pipe shaped portion, so that the suction cup retains its position on the breast even after tilting the cylinder. In addition, the suction cup is inclined about an angle between 30° up to 90° with respect to the longitudinal axis of the pipe shaped portion, in order to keep the pump approximately vertical in the operation. The suction cup can be provided with an adapter or a cladding, respectively, in order to perform an adaptation to a small or flat breast.

From DE 8 702 791 U1, a similar manual breast pump with a pipe-shaped portion slewably arranged in the outer cylinder is known, wherein an insert made of a soft, resilient polymer material is arranged in the suction cup.

The known manually operable breast pumps have the disadvantage that the hollow piston may be tilt inside the cylinder and thus it may block. Further, flowing over of milk can occur on the sealing rings. Milk flowing back from the cylinder into the hollow piston may easily come into contact with the breast or leak out from the suction cup. In order to perform several suction strokes, the suction cup has to be taken away from the breast before pushing in the hollow piston into the cylinder. After pushing in, the suction cup has to be sealingly positioned on the breast again. When the hollow piston is completely thrust into the cylinder, the inclined connection pipe to the suction cup sits closely on the upper brim of the cylinder. As a consequence, it is difficult to hold fast the hollow piston when the cylinder is to be drawn downward for lighting.

The present invention provides a manually operable breast pump with better usage properties.

### BRIEF SUMMARY OF THE INVENTION

The manual breast pump for pumping down breast milk has:
a cylindrical hollow piston with at least one deepening, circulating on the outside, on the lower end with respect to the cylindrical superficies surface of the hollow piston,
a suction cup, which is connected to the upper end of the hollow piston via a passage for fluid,
a soft elastic compensation element between the hollow piston and the bearing surface of the suction cup,
at least one circulating sealing element made of a soft elastic material, inserted into the deepening, and
a cylinder with a closed lower end, in which the hollow piston is axially movable, wherein the superficies surface of the hollow piston is guided on an inner wall of the cylinder, the sealing element is sealingly arranged between the inner wall of the cylinder and the hollow piston, and at least the suction cup is arranged outside of the cylinder.

In the breast pump of the present invention, jamming and blocking of the hollow piston in the cylinder is avoided through the long guideway of the hollow piston on its superficies surface in the cylinder. The sealing element sits in the deepening inside the superficies surface and protrudes above the superficies surface only with a deformable portion, which sits closely on the inside wall of the cylinder on its outside. Due to the accommodation of the sealing element substantially inside the superficies surface, it is possible that the inside diameter of the cylinder is only slightly in excess of the outer diameter of the hollow piston, so that a very good guiding of the hollow piston in the cylinder is achieved. Different orientations of the cylinder when using the breast pump are compensated by the soft elastic compensation element, so that the bearing surface of the suction cup retains its position on the breast of the user. The drawbacks connected with the different orientations of the breast pump towards the user are avoided.

The soft elastic compensation element can be realised in different ways. For instance, it is a flexible pipe or a flexible tube between the upper end of the hollow piston and the passage for fluid of the suction cup.

According to a preferred embodiment, the soft elastic compensation element is an insert made of a soft elastic material, arranged in the suction cup, which is kept sealingly at its brim side on the brim of the great opening of the suction cup and which has a central passage hole through which the suction negative pressure is applied and the breast milk flows down. The flexible insert adapts itself particularly well to the mother's breast and is very comfortable when it is used. In addition, it maintains it's bearing position on the breast when the breast pump is changed in its position due to tilting of the cylinder, and it compensates the change of position by elastic deformation.

The manual breast pump for pumping down breast milk has:
a cylindrical hollow piston with a circulating groove on the lower end,
a suction cup, which is connected to the upper end of the hollow piston via a passage for fluid,
a circulating double lip seal made of a soft elastic material arranged at the lower end of the hollow piston, which has two double lips pointing into different directions with respect to an annular main body, which are each at a time directed in an acute angle to the axis of the hollow piston, wherein the annular main body sits on the bottom of the groove, and
a cylinder with a closed lower end, in which the hollow piston is axially movable, wherein the double lip seal is sealingly arranged between the inner wall of the cylinder and the hollow piston, and at least the suction cup is arranged outside of the cylinder.

In the breast pump of the present invention, the double lip seal provides an effective sealing of the hollow piston with respect to the cylinder when generating the negative pressure for suction, and it prevents an overflow of mother's milk from the cylinder into the clearance between hollow piston and cylinder in a particularly effective manner. The tightness when sucking down is provided by the sealing lip pointing away from the closed end of the cylinder in particular, and the tightness against the overflow of breast milk is provided by the sealing lip pointing towards the closed end of the cylinder in particular.

Advantageously, the breast pump can have the features of one or plural of the already known breast pumps.

The double lip sealing can be shaped in different ways. For instance, it may be formed in one piece with the hollow piston, by injection moulding of a flexible plastic material onto it for instance. According to a preferred embodiment, the double lip seal is a double lip sealing member, which is arranged on a sealing seat of the hollow piston. The sealing member to be installed separately permits exchange upon wear or damage, respectively.

The manual breast pump for pumping down breast milk has:
a cylindrical hollow piston,
a suction cup, which is connected to the upper end of the hollow piston via a passage for fluid,
an insert arranged in the suction cup, which is kept sealingly at its brim side on the brim of the great opening of the suction cup and which has a central passage hole, wherein between suction cup and insert a there is retaining space with at least one further passage for fluid towards the hollow piston,
at least one circulating sealing element made of a soft elastic material on the lower end of the hollow piston, and
a cylinder with a closed lower end, in which the hollow piston is axially movable, wherein the sealing element is sealingly arranged between the inner wall of the cylinder and the hollow piston, and at least the suction cup is arranged outside of the cylinder.

In the breast pump of the present invention, milk flowing back enters the retaining space through the additional passage for fluid between the suction cup and the insert and is collected there. The contact of milk flowing back with the mother's breast and the incommodities and possible contaminations of the breast milk as well as leaking of the breast milk out of the suction cup is avoided. When the breast pump is set on correctly later, the milk flows back into the cylinder and is collected there.

Advantageously, the breast pump can have the features of one or several ones of the already known breast pumps.

The insert can be shaped differently. According to a preferred embodiment, it is substantially funnel-shaped or conical, respectively. With a funnel-shaped insert and a suction cup which is funnel-shaped or conical, respectively, the retaining space can be realised in that the insert has a greater cone angle than the suction cup. According to a further embodiment, the insert is substantially trumpet-shaped. Through the trumpet-shaped realisation, a particularly large retaining space can be provided with a funnel-shaped suction cup.

In principle, the insert can consist of a hard or hard-elastic material. According to a preferred embodiment, the insert consists of a soft elastic material, so that it adapts itself well to the breast and compensates position changes of the cylinder by elastic deformation.

The further passage for fluid can be realised in different ways. For instance, in this, it may be matter of at least one passage opening which surrounds the insert in portions. According to a preferred embodiment, the passage for fluid is an annular gap between the suction cup and the insert.

The manual breast pump for pumping down breast milk has:
a cylindrical hollow piston,
a suction cup, which is connected to the upper end of the hollow piston via a passage for fluid,
a check valve arranged in the wall of the suction cup, which opens at a pressure burden in the suction cup and is otherwise closed,
at least one circulating sealing element made of a soft elastic material on the lower end of the hollow piston, and
a cylinder with a closed lower end, in which the hollow piston is axially movable, wherein the sealing element is sealingly arranged between the inner wall of the cylinder and the hollow piston, and at least the suction cup is arranged outside of the cylinder.

When performing plural suction strokes, the breast pump of the present invention does not need to be set off from the breast. The pressure burden generated when the hollow piston and the cylinder are thrust together is reduced by the check valve. As a consequence, taking off and setting up again of the breast pump at different suction strokes, which makes the use less comfortable, can be avoided. It is not necessary to find a sealing set-on position of the suction cup before a further suction stroke.

Advantageously, the breast pump can have the features of one or several ones of the breast pumps described above.

The check valve can be realised in different ways, as a ball check valve with a spring-tensioned ball as the valve gate, for instance. According to one embodiment, the check valve is a flap valve. This permits particularly simple executions of the check valve. According to a further embodiment, the check valve has an annular enclosure with a flap, articulated on the inside periphery via an elastic bridge, and the enclosure is arranged on a cylindrical seating surface of the suction cup surrounding a perforated bottom, wherein the flap sits close to the perforated bottom in the not deflected condition. This realisation is particularly simple in its construction and user-friendly. In particular, replacement of the check valve in the case of wear or damage is easily possible. According to a further embodiment, the check valve is made in one piece from a soft elastic material.

A manual breast pump for pumping down breast milk has:
a cylindrical hollow piston,
a suction cup, which is connected to the tipper end of the hollow piston via a passage for fluid,
at least one circulating sealing element of a soft elastic material on the lower end of the hollow piston, and
a cylinder with a closed lower end, in which the hollow piston is axially movable, wherein the sealing element is sealingly arranged between the inner wall of the cylinder and the hollow piston, and the axial extensions of the cylinder and the hollow piston are selected such that the lower end of the suction cup projects for at least one thumb width over the upper end of the cylinder when the hollow piston is maximally thrust into the cylinder.

In the breast pump according to the present invention, grasping the hollow piston is facilitated by the at least thumb wide projection of the suction cup over the cylinder when hollow piston and cylinder are maximally thrust together. When starting the pumping down action, the user can hold fast the hollow piston between thumb and index finger or other individual fingers, and pull down the cylinder conveniently. A thumb width corresponds to a distance of about two centimetres.

Advantageously, the breast pump can have the features of one or several ones of the breast pumps described above.

According to one embodiment, the distance of the suction cup from the upper end of the cylinder is at least two centimetres when the hollow piston is thrust maximally into the cylinder. Through this, grasping the cylinder is facilitated further.

In this patent application, with “suction cup” a hollow structure is meant, which tapers towards the one end and has an opening at the other end. The tapering can have different shapes, it may be funnel-shaped or conical, respectively, cup-like or bell-like or otherwise vaulted or stepped, or combine different ones of these shapes. Preferably, the suction cup is shaped and dimensioned such that it can accommodate a female breast without touching a nipple or the areola.

In principle, the suction cup can have the form of a cone. According to one embodiment, the suction cup has a bulged form with respect to a cone, in adaptation to the form of the breast or for the purpose to accommodate the retaining space in the variant of the invention with a retaining space between suction cup and insert, respectively.

The suction cup may be a separate part, which is connected to tile hollow piston via a connection part, a tube or a pipe for instance. According to one embodiment, the suction cup is integrally connected with the hollow piston.

After collecting the breast milk in the cylinder, it may be proceeded with it in different ways. For instance, the breast milk can be filled into a feeding bottle. The cylinder can be used as the container of a drinking bottle. For this purpose, the cylinder has an outside thread on the upper brim according to one embodiment. The outside thread permits to screw up a lid or a drinking teether, respectively.

According to a further embodiment, the cylinder has a pedestal on the lower end, which facilitates the keeping of the filled cylinder. The pedestal may be also used for grasping the cylinder in the suction stroke. According to a preferred embodiment, the pedestal is oval, by which the use of the pedestal as a handle is favoured.

In principle, the cylinder and the hollow piston can be made from a transparent material. According to one embodiment, the cylinder and/or the hollow piston is/are at least partially transparent. The cylinder and/or the hollow piston being transparent facilitates the observation of the pumping down process and the filling amount. According to a further embodiment, there is a scale with volume indications in a transparent region of the cylinder, which facilitates the determination of the filling amount.

Different materials can be taken into consideration for the hollow piston and/or the suction cup. Preferably, these materials are hard or hard-elastic. According to one embodiment the hollow piston and/or the cylinder and/or the suction cup are made from PP and/or PC and/or PE and/or ABS and/or PA and/or PES and/or glass. In particular, at least one thermoplastic material may be used. The mentioned materials offer advantages in the manufacture, are easy to clean and are advantageous in hygienic aspects.

In principle, the suction cup can be set directly onto the mother's breast. The insert already mentioned as elastic compensation element and as limitation of a retaining space is used in the further breast pumps according to a further embodiment, with the already mentioned advantages. According to one embodiment, the insert is funnel-shaped or trumpet-shaped, which favours the sealing fit on the mother's breast and supports the build-up of a retaining space between insert and suction cup, respectively.

According to one embodiment, the insert is soft elastic. According to a further embodiment, the insert has plural knobbles on the outer side, which generate a massage effect by the natural movement process and the movement to and from during pumping down.

According to one embodiment, the insert is made of natural rubber or synthetic rubber (silicone rubber in particular). An insert made of rubber is particularly soft, elastic, easy to clean and hygienically acceptable.

According to a realisation which is particularly simple in its construction, the sealing element is an O-ring. Plural O-rings may also be used. Further, an O-ring can be combined with a single or double lip seal.

According to a further embodiment the sealing element and/or the check valve is/are made of natural rubber or synthetic rubber (silicone rubber in particular). A sealing element made of rubber is a particularly elastic, easy to clean and hygienically acceptable material.

### BRIEF DESCRIPTION OF THE SEVERAL VIEW OF THE DRAWINGS

The present invention will be explained in more detail in the following by means of the attached drawings of examples of its realisation. The drawings show:

FIG. 1 a manual breast pump with trumpet-shaped inserts, check valve and double lip sealing member in a cross section;

FIG. 2 the same breast pump cut in the longitudinal direction, and in a perspective view;

FIG. 2a a check valve of the same breast pump in a front view;

FIG. 3 the same breast pump in a side view;

FIG. 4 hollow piston with suction cup of the same breast pump in a side view;

FIG. 5 a breast pump with transparent cylinder in a side view;

FIG. 6 the same breast pump in a longitudinal section;

FIG. 7 breast pump with transparent cylinder and shortened insert in the longitudinal section;

FIG. 8 breast pump with transparent cylinder and sealing O-ring in a perspective side view; and

FIG. 9 the same breast pump longitudinally cut in the same perspective view.

### DETAILED DESCRIPTION OF THE INVENTION

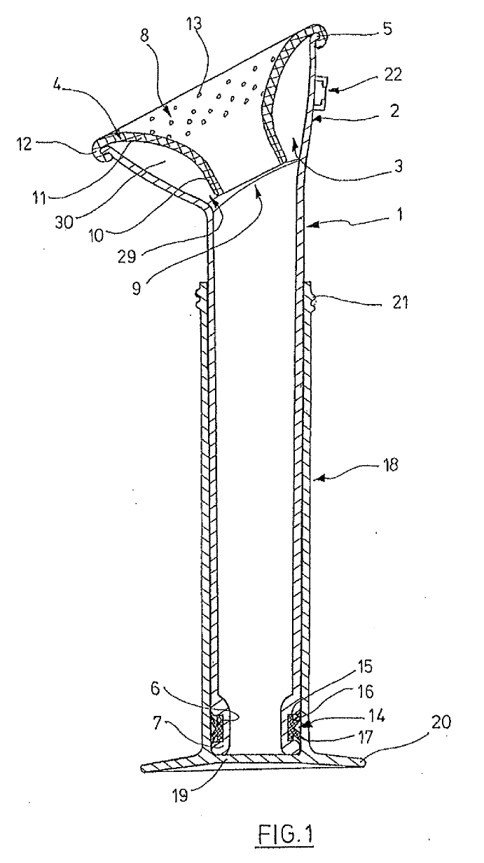

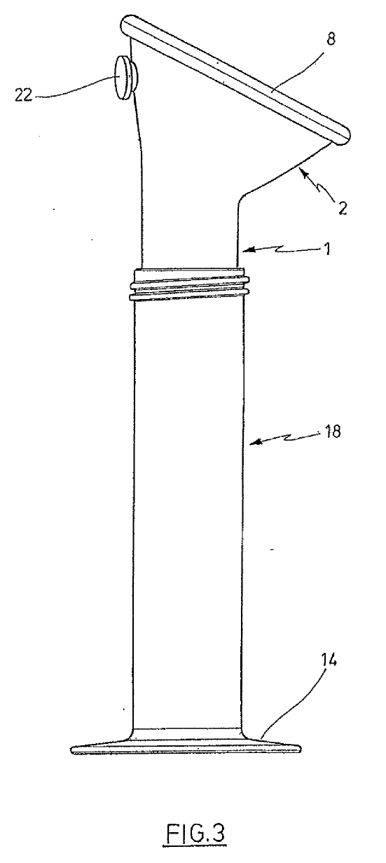

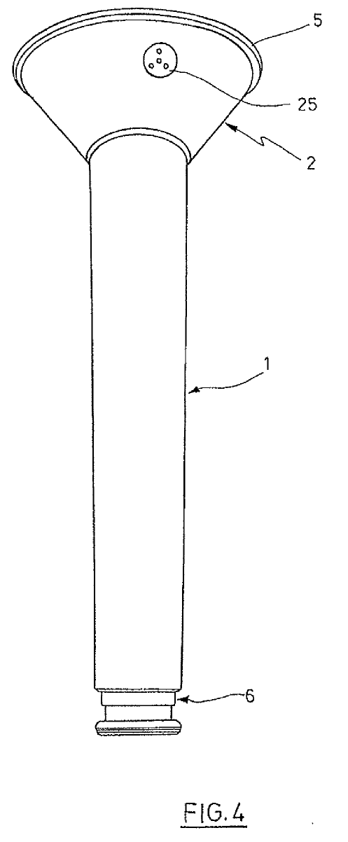

The breast pump according to FIG. 1 to 4 has a cylindrical hollow piston 1, which is connected in one piece on its upper end with a suction cup 2. The suction cup 2 is a hollow rotational body, the central axis of which is inclined in an acute angle with respect to the central axis of the hollow piston 1. In the connection cross section to the hollow piston 1, the suction cup 2 has a passage for fluid 3 towards the hollow piston 1.

The suction cup 2 has an essentially funnel-shaped form. Its cross section enlarges gradually, departing from the passage for fluid 3 up to a great, circular opening 4 on the outer end. The opening 4 is surrounded by a brim bead 5. The shape of the suction cup 2 is somewhat bulged towards the outside with respect to a cone.

The hollow piston 1 has a deepening in the form of a circulating groove 6 on the lower end. Adjoining to the lower end, the hollow piston 1 has a portion 7 with enhanced wall thickness, which accommodates the groove 6.

The hollow piston 1 and the suction cup 2 are preferably made integrally from PP or PC or glass.

An essentially funnel-shaped insert 8 is set into the suction cup 2. The shape of the insert 8 can be more accurately described as trumpet-shaped. The insert 8 has a central passage hole 9, to which a short cylindrical portion 10 adjoins. A region 11 gradually enlarging towards the outside departs from the cylindrical portion 10. The former has a back-bent brim 12, which is snapped over the brim bead 5 and thus is sealingly fixed on the same.

On the outer side, the insert 8 has a plurality of projecting knobbles 13.

The insert 8 is preferably integrally made of silicone rubber.

In the groove 6, a double-lip sealing member 14 is arranged. On an annular main body 15 sitting on the bottom of the groove 6, the same has two circulating lips 16, 17, which are inclined about acute angles in different directions with respect to the annular main body 15. The double lip sealing member 14 is preferably made in one piece from silicone rubber.

The hollow piston 1 is thrust into a cylinder 18, which has a slight oversize in the inside with respect to the cylindrical superficies surface of the hollow piston 1. The oversize is selected such that the hollow piston 1 is easily movable inside of the cylinder 18, yet is guided in the cylinder 18 such that it does not jam therein.

The two lips 16, 17 of the double lip sealing member 14 sit sealingly close to the inner wall of the cylinder 18.

On the downside, the cylinder 18 is closed by a bottom 19. The bottom 19 is a part of a pedestal 20, which projects towards the outside with respect to the cylinder 18.

Further, the cylinder 18 has an outside thread 21 on its upper brim.

The cylinder 18 is preferably integrally made from PP, PC or glass.

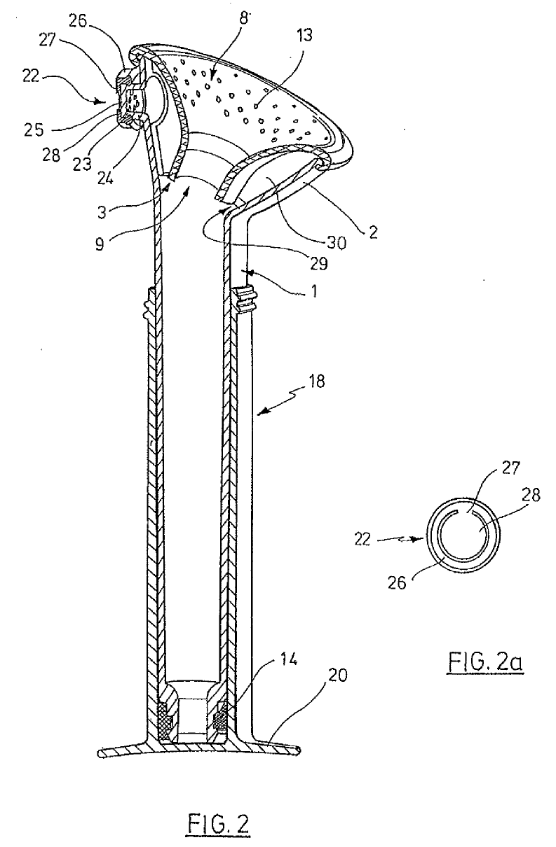

The suction cup 2 bears a check valve 22. The same is positioned on a cylindrical seat surface 23 of a sleeve-shaped neck 24, which is arranged on the outer surface of the suction cup 2 (compare FIG. 2). The neck 24 has a perforated bottom 25.

The check valve 22 has an annular enclosure 26, which sits closely on the seat surface 23. On the inner brim of the enclosure, a flap 28 is articulated via a flexible bridge 27, which sits closely on the perforated bottom 25. When there is a pressure burden, the flap 28 is lifted off from the perforated bottom 25.

According to another embodiment, the valve has a valve gate on a membrane. The membrane is supported on the suction cup at the outside brim, and a sealing seat in a passage hole of the suction cup is associated to the valve gate.

When there is a negative pressure or normal pressure in the suction cup, the valve gate sits closely on the sealing seat and the passage hole is closed. Upon pressure burden, membrane and valve gate are deflected und the passage hole opens itself.

The lengths of the hollow piston 1 and of the cylinder 18 are dimensioned such that the suction cup 2 projects about at least one thumb width over the upper brim of the cylinder 18 when the hollow piston 1 is completely thrust into the cylinder 18, so that the lower end of the hollow piston 1 sits closely on the bottom 19 (compare FIG. 1 to 3).

In the use, the flexible insert 8 is set onto the female breast. The user holds fast the hollow piston 1 between thumb and index finger, between suction cup 2 and upper end of the cylinder 18, and pulls the cylinder 18 downward with the other hand. In this, milk flows into the cylinder 18. The double lip seating member 14 secures a high negative pressure when sucking and prevents the intrusion of milk into the gap between hollow piston 1 and cylinder 18. A pressure burden when thrusting back the cylinder 18 can escape via the check valve 22. In the case that the suction cup 2 is used at backward inclined waist or in recumbence, respectively, breast milk flowing back may reach the retaining space 30 between insert 8 and suction cup 2 through an annular gap 29 at the most, so that a contact with the mother's breast and leaking out are avoided.

After use, the hollow piston 1 can be pulled out and a not shown lid or a not shown drinking teether can be screwed on the outside thread 21.

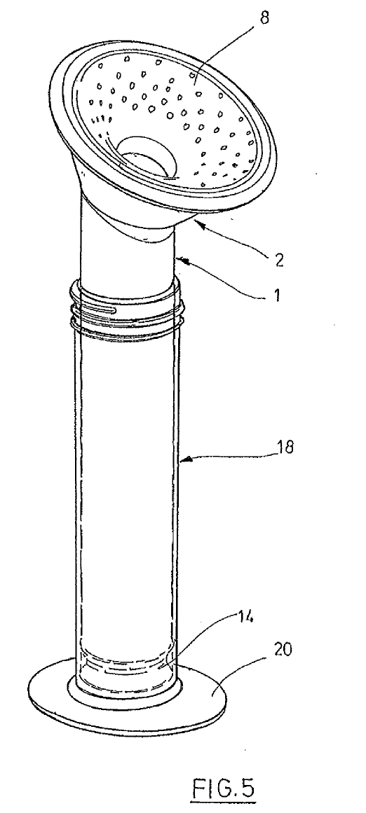

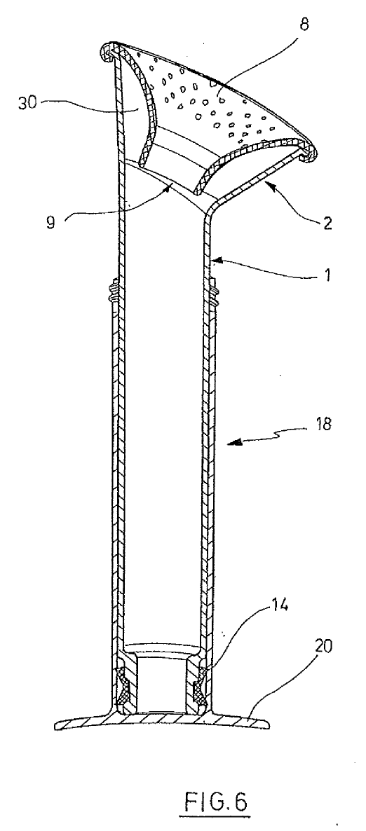

The realisation example according to FIG. 5 and 6 differs from that one described above through a transparent cylinder 18, which permits to observe the amount of milk aspirated in. Preferably, the cylinder 18 is provided with a not shown scale in the vertical direction. As the case may be, the scale is provided with volume indications.

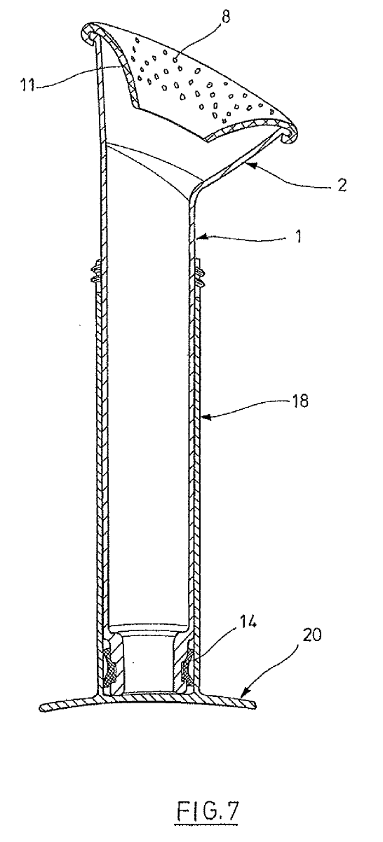

The realisation of FIG. 7 differs from that one above through the execution of the insert 8. This insert 8 does not have the cylindrical portion 10, so that the small opening 9 adjoins directly to the enlarging portion 11. This realisation without “chimney” can be advantageous in certain applications. In addition, there is no check valve 22.

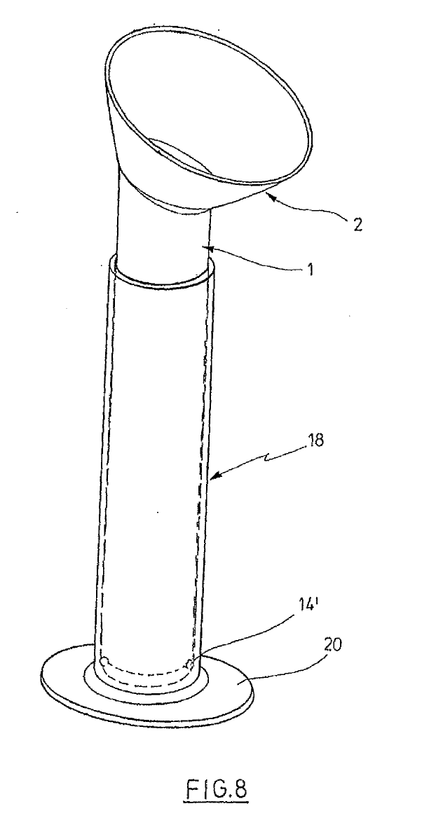

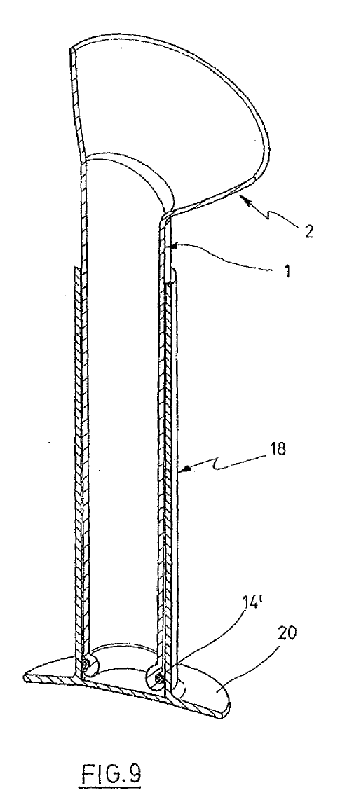

The realisation of FIGS. 8 and 9 differs from that one according to FIGS. 5 and 6 in that a sealing O-ring 14′ is arranged in a suitably shaped annular groove 6′ on the lower end of the hollow piston 1. Furthermore, the suction cup 2 has no insert 8 and the cylinder 18 has no outside thread 21. In this very simple variant, however, the suction cup 2 projects for at least one thumb width over the cylinder 18.

While this invention may be embodied in many different forms, there are described in detail herein a specific preferred embodiment of the invention. This description is an exemplification of the principles of the invention and is not intended to limit the invention to the particular embodiment illustrated.

In the following description of different examples of realisation, the same or essentially coincident component parts are provided with the same reference numerals.

1. A breast pump for pumping down breast milk, with
a cylindrical hollow piston (1) with at least one deepening (6) circulating on the outside, on the lower end with respect to the cylindrical superficies surface of the hollow piston (1),
a suction cup (2), which is connected to the upper end of the hollow piston (1) via a passage for fluid (3),
a soft elastic compensation element (8) between the hollow piston (1) and the bearing surface of the suction cup (2),
at least one circulating sealing element (14) made of a soft elastic material, inserted into the deepening (6), and
a cylinder (18) with a closed lower end, in which the hollow piston (1) is axially movable, wherein the superficies surface of the hollow piston (1) is guided on an inner wall of the cylinder (18), the sealing element (14) is sealingly arranged between the inner wall of the cylinder (18) and the hollow piston (1), and at least the suction cup (2) is arranged outside of the cylinder (18).

2. A breast pump according to claim 1, wherein the elastic compensation element is an insert (8) made of a soft elastic material, arranged in the suction cup (2), which is kept sealingly at its brim side on the brim of the great opening (4) of the suction cup (2), and which has a central passage hole (9), through which the negative pressure for suction is applied and the breast milk flows down.

3. A manual breast pump for pumping down breast milk, with
a cylindrical hollow piston (1) with a circulating groove (6) on the lower 19 end,
a suction cup (2), which is connected to the upper end of the hollow piston (1) via a passage for fluid (3),
a circulating double lip seal (14) made of a soft elastic material arranged at the lower end of the hollow piston (1), which has two double lips (16, 17) pointing into different directions with respect to an annular main body (15), which are each at a time directed in an acute angle to the axis of the hollow piston (1), wherein the annular main body (15) sits on the bottom of the groove (6), and
a cylinder (18) with a closed lower end, in which the hollow piston (1) is axially movable, wherein the double lip seal (14) is sealingly arranged between the inner wall of the cylinder (18) and the hollow piston (1), and at least the suction cup (2) is arranged outside of the cylinder.

4. (canceled)

5. A breast pump according to claim 3, wherein the double lip seal (14) is a double lip sealing member, which is arranged on a sealing seat of the hollow piston (1).

6. A manual breast pump for pumping down breast milk, with
a cylindrical hollow piston (1)
a suction cup (2), which is connected to the upper end of the hollow piston (1) via a passage for fluid (3),
an insert (8) arranged in the suction cup (2), which is kept at its brim side on the brim of the great opening (4) of the suction cup (2) and which has a central passage hole (9), wherein between suction cup (2) and insert (8) there is a retaining space (30) with at least one further passage for fluid (29) towards the hollow piston (1),
at least one circulating sealing element (14) made of a soft elastic material on valve.

7. A breast pump according to claim 6.

8. A breast pump according to claim 6, wherein the additional passage for fluid (29) is an annular gap between the suction cup (2) and the insert (8).

9. A manual breast pump for pumping down breast milk, with
a cylindrical hollow piston (1)
a suction cup (2), which is connected to the upper end of the hollow piston (1) via a passage for fluid (3),
a check valve (22) arranged in the wall of the suction cup (2), which opens at a pressure burden in the suction cup (2) and is otherwise closed,
at least one circulating sealing element (14) made of a soft elastic material on the lower end of the hollow piston (1), and
a cylinder (18) with a closed lower end, in which the hollow piston (1) is axially movable, wherein the sealing element (14) is sealingly arranged between the inner wall of the cylinder (18) and the hollow piston (1), and at least the suction cup (2) is arranged outside of the cylinder (18).

10. A breast pump according to claim 9.

11. A breast pump according to claim 9, wherein the check valve (22) is a flap valve.

12. A breast pump according to claim 11, wherein the check valve has an annular enclosure (26) with a flap (28), articulated on the inside periphery via an elastic bridge (27), and the enclosure is arranged on a cylindrical seating surface (23) of the suction cup (2) surrounding a perforated bottom (25), wherein the flap (28) sits close to the perforated bottom (25) in the not deflected condition.

13. A breast pump according to claim 12, wherein the check valve (22) is made in one piece from a soft elastic material.

14. A manual breast pump for pumping down breast milk, with
a cylindrical hollow piston (1),
a suction cup (2), which is connected to the upper end of the hollow piston (1) via a passage for fluid (3),
at least one circulating sealing element (14) made of a soft elastic material on the lower end of the hollow piston (1), and
a cylinder (18) with a closed lower end, in which the hollow piston (1) is axially movable, wherein the sealing element (14) is sealingly arranged between the inner wall of the cylinder (18) and the hollow piston (1), and the axial extensions of the cylinder (18) and the hollow piston (1) are selected such that the lower end of the suction cup (2) projects for at least one thumb width over the upper end of the cylinder (18) when the hollow piston (1) is maximally thrust into the cylinder (18).

15. (canceled)

16. A breast pump according to claim 14, wherein the distance of the suction cup (2) from the upper end of the cylinder (18) is at least two cm when the hollow piston (1) is maximally thrust into the cylinder (18).

17. A breast pump according to claim 1, wherein the suction cup (2) has a bulged form with respect to a cone.

18. A breast pump according to claim 1, wherein the suction cup (2) is integrally connected with the hollow piston (1).

19. A breast pump according to claim 1, wherein the cylinder (18) has an outside thread (21) on the upper brim.

20. A breast pump according to claim 1, wherein the cylinder (18) has a pedestal (20) on the lower end.

21. A breast pump according to claim 20, wherein the pedestal (20) is oval.

22. A breast pump according to claim 1, wherein the cylinder (18) and/or the hollow piston (1) is/are at least partially transparent.

23. A breast pump according to claim 1, wherein the cylinder (18) has a scale.

24. A breast pump according to claim 1, wherein the hollow piston (1) and/or the cylinder (18) and/or the suction cup (2) are made from PP and/or PC and/or PE and/or ABS and/or PA and/or PES and/or glass.

25. A breast pump according to claim 1, wherein an insert (8) is arranged in the suction cup (2), which is kept sealingly at the brim side on the brim of the great opening of the suction cup (2) and which has a central passage hole (9).

26. A breast pump according to claim 1, wherein the insert (8) is funnel-shaped or trumpet-shaped.

27. A breast pump according to claim 1, wherein the insert (8) is soft elastic.

28. A breast pump according to claim 1, wherein the insert (8) has plural knobbles (13) on the outer side.

29. A breast pump according to claim 1, wherein the insert (8) is made of natural rubber or synthetic rubber.

30. A breast pump according to claim 1, wherein the sealing element (14) is an O-ring.

31. A breast pump according to claim 1, wherein the sealing element (14) and/or the check valve (22) is/are made of natural rubber or synthetic rubber.
